(19) United States
(12) Patent Application Publication
(10) Pub. No.: US 2025/0134785 A1
(43) Pub. Date: May 1, 2025

(54) Composition Comprising Microcapsules for Solid Consumer Products

(71) Applicant: Symrise AG, Holzminden (DE)

(72) Inventors: Julian Alexander GEORGI, Kassel (DE); Thorsten BÖDDEKER, Brakel (DE); Nelli AHRENS, Reinbeck (DE); Jonas MEISEL, Uslar (DE); Antonia Franziska TRAPP, Holzminden (DE); Jörn WIEDEMANN, Holzminden (DE)

(21) Appl. No.: 18/837,180

(22) PCT Filed: Feb. 11, 2022
(86) PCT No.: PCT/EP2022/053405

§ 371 Date: Aug. 8, 2024

(51) Int. Cl. A61K 8/04; A61K 9/20; A61Q 13/00; A61Q 15/00; A61Q 19/10; C11D 3/37; C11D 3/38; C11D 3/50; C11D 11/00; C11D 17/00

(52) U.S. Cl. CPC A61K 8/04; A61K 9/2081; A61Q 13/00; A61Q 15/00; A61Q 19/10; C11D 3/3707; C11D 3/38; C11D 3/505; C11D 11/0082; C11D 17/0086

(57) ABSTRACT

The present invention relates to the field of perfumed particles in particular for solid consumer product formulations such as textile care products. More specifically, the present invention relates to a composition comprising or consisting of at least one pastille, wherein said pastille comprises or consists of at least one microcapsule as well as a polymer matrix embedding said at least one microcapsule as well as to a composition additionally comprising or consisting of at least one adhesion agent in said matrix. Moreover, the present invention relates to a process for the preparation of a corresponding composition and its use for the preparation of consumer products as well as consumer products as such comprising or consisting of the composition.

### FIELD OF THE INVENTION

The present invention relates to the field of perfumed particles in particular for solid consumer product formulations such as textile care products. More specifically, the present invention relates to a composition comprising or consisting of at least one pastille, wherein said pastille comprises or consists of at least one microcapsule as well as a polymer matrix embedding said at least one microcapsule as well as to a composition additionally comprising or consisting of at least one adhesion agent in said matrix. Moreover, the present invention relates to a process for the preparation of a corresponding composition and its use for the preparation of consumer products as well as consumer products as such comprising or consisting of the composition.

### BACKGROUND OF THE INVENTION

Nowadays many consumer products such as detergents, fabric softeners, washing powders, deodorants, lotions, etc. are perfumed with fragrance materials or contain cosmetic ingredients to deliver a specific effect.

Unfortunately, often, for example, the fragrant substances or cosmetic ingredients of such products interact with other components of the product formulation, or the more volatile components of the perfume formulation tend to evaporate prematurely. Consequently, this leads to an undesired change and/or decrease of the fragrance impression or premature “use” of the cosmetic ingredient over time or might even cause disadvantageous reactions with the other ingredients of the product formulation resulting in a reduced product quality and/or stability. In order to prevent possible interactions of the fragrances or other active components with the other ingredients of the product or, for example, to prevent the volatilization of the fragrances and thus not to distort or reduce the desired olfactory impression, the fragrances or other active ingredients can be added to the formulation in encapsulated form. In this way, for example the desired olfactory impression can be guaranteed based on a targeted release of the active component. Moreover, interactions between the product components can be reduced allowing for improved product quality and storage stability.

Therefore, a broad variety of consumer products comprise encapsulated active ingredients such as fragrances in their formulations. For example, textile care formulations such as liquid detergents or fabric softener comprise a plurality of such perfumed microcapsules. Additionally, said formulations require the incorporation of preservatives in order to increase their stability and durability such as methylisothiazolinone (MIT) which can cause allergic reactions or skin irritations, while powdered detergents do not require the addition of preservatives and cause a reduced environmental pollution. However, the incorporation of microcapsules into powdered formulations is considerably aggravated as a mixing of the microcapsule component and the other detergent components leads to breakage of the fragile microcapsules resulting in a loss of for example olfactory performance. It is particularly difficult to produce microcapsules that have both good stability and good release properties. The ability of the capsules to retain the active ingredient and thus to prevent the loss of volatile components depends in particular on the stability of the capsules in the product base. However, very stable microcapsules, e.g. capsules having a comparably thick capsule wall, usually tend to exhibit a low performance as the break of the microcapsules and thus the release of the active ingredient(s) is hindered. However, if they are too unstable, they are already destroyed during storage or result in a leakage of the active ingredient and do not perform either. Thus, an increase of the capsule wall, i.e. a multi-layered microcapsule shell or a thicker shell, respectively, likewise result in a decreased performance as the targeted release of the active ingredient is aggravated and said microcapsules usually require a complex multi-step preparation process.

In addition, there is a new and increasing trend towards solid consumer products such as solid hair care products, soaps and solid body wash in view of the public's increased ecological awareness (less waste, less preservatives, etc.), so that solid consumer products will become more and more relevant not only in the cosmetic industry but also in other

Consequently, there is need to improve the efficient incorporation of actives such as perfuming ingredients into solid consumer product formulations, which however, simultaneously allow for an efficient and targeted release of said active.

### SUMMARY OF THE INVENTION

The present invention was made in view of the disadvantages described above. To solve the above-mentioned drawbacks, the present invention provides a compositions and a method for the preparation thereof, allowing for the efficient incorporation of active ingredients into solid consumer product formulations for the preparation of consumer products.

Thus, in a first aspect the present invention relates to composition comprising or consisting of at least one pastille, wherein said pastille (based on the total weight of the pastille) comprises or consists of:
(a) from 0.5% to 10% by weight of at least one microcapsule encapsulating an active ingredient,
   wherein said active ingredient is preferably a fragrance or fragrance mixture; and
(b) from 92% to 98% by weight of a polymer,
   wherein said polymer is preferably polyethylene glycol.

In a preferred embodiment of the composition, the polymer is preferably polyethylene glycol.

According to a second aspect the present invention relates to a composition comprising or consisting of at least one pastille, wherein said pastille (based on the total weight of the pastille) comprises or consists of:
(a) from 0.5% to 10% by weight of at least one microcapsule encapsulating an active ingredient,
   wherein said active ingredient is preferably a fragrance or fragrance mixture;
(b) from 60% to 80% by weight of at least one polymer,
   wherein said polymer is preferably polyethylene glycol; and
(c) from 2% to 32% by weight of at least one adhesion agent,
   wherein said adhesion agent is preferably selected from the group consisting of fatty acids, fatty alcohols, fatty acid esters or mixtures thereof, such as waxes.

In a preferred embodiment of said composition, the polymer is preferably polyethylene glycol, and the adhesion agent is selected from the group consisting of animal and/or vegetable waxes, and is preferably beeswax.

According to a third aspect the present invention relates to a process for the preparation of a composition comprising or consisting of a plurality of pastilles, comprising the following steps of:
(i) providing at least one microcapsule or an aqueous dispersion comprising at least one microcapsule;
(ii) melting of at least one polymer and optionally an adhesion agent;
(iii) mixing the at least one microcapsule into the polymer melt of step (ii);
(iv) forming pastilles by dripping the mixture of step (iii); and
(v) optionally cooling;
   optionally further comprising the step of adding at least one plasticizer and/or a structuring agent in or after step (ii) or (iii).

Additionally, in a further aspect of the present invention relates to compositions prepared according to the inventive process.

Finally, the present invention relates to the use of a composition according to the invention for the preparation of consumer products and formulations thereof as well as consumer products and formulations thereof as such comprising or consisting of the composition according to the present invention.

That is, the present inventors have found that the compositions according to the invention allow for an efficient incorporation of active ingredients in encapsulated form which show excellent release properties (performance) into a broad range of solid consumer product formulations and consumer products as such. Thereby, the polymeric matrix serves as a buffer protecting the fragile microcapsules during the preparation of the final consumer product (formulation) from mechanical, chemical and/or thermal influences without negatively influencing the performance of the microcapsules within the consumer product (formulation). Based thereon the present dosage form allows for an efficient incorporation of active ingredients via microcapsules in solid consumer product formulations while simultaneously allowing for an efficient and targeted release of said active ingredients thus overcoming the drawbacks of the state of the art. The fragile microcapsules are embedded in a protecting matrix which even withstands harsh chemical and physical conditions such as increased temperatures or acidic or basic conditions.

The present problem is solved by the objects of the independent patent claims. Preferred embodiments are apparent from the wording of the dependent patent claims as well as the following description.

All percentages are by weight unless otherwise indicated. Numeric examples given in the form “x to y” include the values given. When multiple preferred numeric ranges are specified in this format, all ranges created by combining the various endpoints are also included.

The terms “at least one” or “one or more” as used herein refers to 1 or more, for example 2, 3, 4, 5, 6, 7, 8, 9 or more.

The term “and/or” expresses that a linkage exists, or an alternative is provided.

### BRIEF DESCRIPTION OF DRAWINGS

FIG. 1 shows the results of sensory evaluation of the performance (i.e. release properties) of different samples of laundry soap compared to samples comprising the composition according to the present invention comprising melamine-formaldehyde-based core-shell microcapsules and polyethylene glycol.

FIG. 2 shows the results of sensory evaluation of the performance (i.e. release properties) of different samples of laundry soap compared to samples comprising the composition according to the present invention comprising melamine-formaldehyde-based core-shell microcapsules, polyethylene glycol and beeswax.

FIG. 3 shows the results of sensory evaluation of the performance (i.e. release properties) of different samples in tabletted form compared to samples comprising the composition according to the present invention comprising melamine-formaldehyde-based core-shell microcapsules and polyethylene glycol.

FIG. 4 shows the results of sensory evaluation of the performance (i.e. release properties) of different samples in tabletted form compared to samples comprising the composition according to the present invention comprising melamine-formaldehyde-based core-shell microcapsules, polyethylene glycol and beeswax.

FIG. 5 shows the results of sensory evaluation of the performance (i.e. release properties) of different samples of powder detergents compared to samples comprising the composition according to the present invention comprising melamine-formaldehyde-based core-shell microcapsules and polyethylene glycol.

FIG. 6 shows the results of sensory evaluation of the performance (i.e. release properties) of different samples of powder detergents compared to samples comprising the composition according to the present invention comprising melamine-formaldehyde-based core-shell microcapsules, polyethylene glycol and beeswax.

FIG. 7 shows the results of sensory evaluation of the performance (i.e. release properties) of different samples of laundry soap compared to samples comprising the composition according to the present invention comprising biodegradable core-shell microcapsules and polyethylene glycol.

FIG. 8 shows the results of sensory evaluation of the performance (i.e. release properties) of different samples of laundry soap compared to samples comprising the composition according to the present invention comprising biodegradable core-shell microcapsules, polyethylene glycol and beeswax.

FIG. 9 shows the results of sensory evaluation of the performance (i.e. release properties) of different samples of powder detergents compared to samples comprising the composition according to the present invention comprising isocyanate-based core-shell microcapsules and polyethylene glycol.

FIG. 10 shows the results of sensory evaluation of the performance (i.e. release properties) of different samples of powder detergents compared to samples comprising the composition according to the present invention comprising isocyanate-based core-shell microcapsules, polyethylene glycol and beeswax.

FIGS. 11A (left) and 11B (right) show the homogeneous distribution of the pastilles according to the present invention having a particle size of 2 mm in a solid laundry soap as well as the pastilles as such, respectively. The tablet size was 3.3 cm.

FIGS. 12A (left) and 12B (right) show the inhomogeneous distribution of the pastilles according to the present invention having a particle size of 5 mm in a solid laundry soap as well as the pastilles as such, respectively. The tablet size was 3.3 cm.

FIGS. 13A (left) and 13B (right) show exemplary pastilles according to the composition of the present invention.

### DETAILED DESCRIPTION OF THE INVENTION

Hereinafter, the present invention is described in more detail.

In a first aspect the present invention relates to a composition comprising or consisting of at least one pastille, wherein said pastille comprises or consists of:
(a) from 0.5% to 10% by weight based on the total weight of the pastille of at least one microcapsule encapsulating an active ingredient,
   wherein said active ingredient is preferably a fragrance or fragrance mixture; and
(b) from 92% to 98% by weight based on the total weight of the pastille of a polymer,
   wherein the polymer is preferably polyethylene glycol (PEG).

Optionally, in a further preferred embodiment the pastille of the composition according to the first aspect further comprises or consists of:
(c) from 0.5% to 10% by weight based on the total weight of the pastille of at least one plasticizer, wherein said plasticizer is preferably water or polyethylene glycol.

In a second aspect the present invention relates to a composition comprising or consisting of at least one pastille, wherein said pastille comprises or consists of:
(a) from 0.5% to 10% by weight based on the total weight of the pastille of at least one microcapsule encapsulating an active ingredient,
   wherein said active ingredient is preferably a fragrance or fragrance mixture;
(b) from 60% to 80% by weight based on the total weight of the pastille of at least one polymer, preferably polyethylene glycol; and
(c) from 2% to 32% by weight of based on the total weight of the pastille at least one adhesion agent,
   wherein said adhesion agent is preferably selected from the group consisting of fatty acids, fatty alcohols, fatty acid esters or mixtures thereof, such as waxes.

According to a further preferred embodiment, the pastille of the composition according to the second aspect further optionally comprises or consists of:
(d) from 0.5% to 10% by weight based on the total weight of the pastille of at least one plasticizer,
   wherein said plasticizer is preferably water or polyethylene glycol; and/or
(f) from 0.1% to 10% by weight based on the total weight of the pastille of a structuring agent,
   wherein said structuring agent is preferably selected from the group consisting of esters, such as isopropyl myristate or other emulsifiers.

### Microcapsules and Active Ingredients

Microcapsules are particles comprising a core and a wall material surrounding the core, wherein the core may be a solid, liquid or gaseous substance or substance mixture surrounded by a polymeric dense, permeable or semi-permeable wall material. The wall is usually formed by precipitation of the polymers upon emulsification and coacervation or interfacial polymerization. The core is also referred to as the inner phase. Other names used for the wall include outer phase, shell or coating. The shell wall can comprise one or more layers and can comprise an additional coating with a coating substance. The diameter of the microcapsules typically varies in the range of 1 to 1,000 μm. The wall thickness is typically 0.5 to 150 μm but can be varied in the range of 5·10⁻⁹ m to 5·10⁻⁶ m. Typically loadings of 25 to 95 wt.-% with core material, but also those of 1 to 99 wt.-% are possible.

For example, hydrophobic active ingredients, such as fragrances or aroma substances, can be easily incorporated into numerous and diverse application formulations through encapsulation.

Based on their properties, microcapsules are used in the printing industry, food industry (vitamins, flavours, plant extracts, enzymes, microorganisms), agricultural chemistry (fertilizers, pesticides), feed industry (minerals, vitamins, enzymes, drugs, microorganisms), pharmaceutical industry, detergent industry, and cosmetic industry, among others.

A variety of capsule wall or coating materials are known for the manufacture of microcapsules. The capsule wall can be made of either natural, semi-synthetic or synthetic materials. Natural shell materials include gum arabic, agar-agar, agarose, maltodextrins, alginic acid or its salts, e.g., sodium alginate or calcium alginate, fats and fatty acids, cetyl alcohol, collagen, chitosan, lecithins, gelatin, albumin, shellac, polysaccharides such as starch or dextran, polypeptides, protein hydrolysates, sucrose, and waxes. Semisynthetic capsule wall materials include chemically modified celluloses, especially cellulose esters, and cellulose ethers, e.g., cellulose acetate, ethyl cellulose, hydroxypropyl cellulose, hydroxypropyl methyl cellulose, and carboxymethyl cellulose, as well as starch derivatives, in particular starch ethers and starch esters. Synthetic shell materials are, for example, polymers such as polyacrylates, polyamides, polyvinyl alcohol or polyvinylpyrrolidone.

Depending on the type of capsule wall material and manufacturing process, microcapsules with different properties in terms of diameter, size distribution, and physical and/or chemical properties are formed in each case.

The microcapsules according to component a) of the composition according to the first aspect can be any of those known to the person skilled in the art.

Preferably, the microcapsules used within the scope of the present invention are such microcapsules, wherein the shell of the at least one microcapsule comprises or consists of a shell material selected from the group consisting of: sol-gel polymer (e.g., silica), polysiloxane, polyacrylate, polyacrylamide, poly(acrylate-co-acrylamide), polyurea, polyurethane, polyamide, polypeptide, polysulfonate, polysaccharide, polyphenolic polymers, poly(melamine-formaldehyde), poly(resorcinol-formaldehyde), poly(urea-formaldehyde), poly(melamine-urea), or combinations thereof.

The composition according to the first and second aspect of the present invention comprises at least one of said microcapsules, i.e. either a single of the above mentioned types of microcapsules is embedded in the polymer matrix of the invention, or two or more microcapsules up to a plurality of microcapsules can be embedded in the polymer matrix. Thereby, all microcapsules can be of the same type, i.e. all microcapsules are for example poly(melamine-formaldehyde)-based, or some of the microcapsules of component a) are for example poly(melamine-formaldehyde)-based and others are polyurea-based, i.e. component a) can comprise or consist of a mixture of different types of microcapsules being based on different encapsulation materials/shell materials.

Preferably, however, the microcapsules according to component a) of the first and/or second aspect of the present invention are selected from the following group of capsule types consisting of: poly(melamine-formaldehyde)-based microcapsules and isocyanate-based microcapsules such as polyurea-based and/or polyurethane-based microcapsules which are preferably biodegradable.

Biodegradable microcapsule-technologies are known to those skilled in the art and are particularly suitable within the context of the present invention in terms of environmental sustainability. Examples of such biodegradable microcapsules are some isocyanate-based microcapsules or polysaccharide-based microcapsules.

The biodegradation of the microcapsule slurry in the environment involves the biological breakdown of the polymeric shell as per the invention. Measurement of biological activity or decomposition of the shell material could be determined under different environments in soil, ocean water, or sludge notably under OECD guidelines. The OECD tests which can be used to determine the ready biodegradability of organic chemicals include the six test methods described in the OECD Test Guidelines No. 301 A-F: DOC Die-Away Test (TG 301 A), CO₂ Evolution Test (TG 301 B), Modified MITI Test (I) (TG 301 C), Closed Bottle Test (TG 301 D), Modified OECD Screening Test (TG 301 E) and Manometric Respirometry Test (TG 301 F). More details can be found in the official OECD Guideline for the testing of chemicals: OECD (2006), Revised Introduction to the OECD Guidelines for Testing of Chemicals, Section 3, OECD Guidelines for the Testing of Chemicals, Section 3, OECD Publishing, Paris.

Within the context of the present invention a microcapsule is considered to be biodegradable if the requirements according to the OECD 301 F biodegradability standard are fulfilled (standardized OECD 301 F test procedure: manometric respirometry test). According to OECD 301 F, a microcapsule is considered readily biodegradable if more than 60% of the wall material has degraded after 28 days.

Consequently, in the composition according to the present invention, the at least one microcapsule is preferably biodegradable according to OECD 301 F.

In the context of the present invention, microcapsules are understood to be microparticles comprising at least one or more active ingredient(s) as core material inside the capsule and which are enclosed by a capsule shell or capsule wall as specified above. As active ingredient to be encapsulated according to the invention, basically any material suitable for inclusion in microcapsules can be considered in the process according to the invention. Preferably, hydrophobic or lipophilic, i.e., water-insoluble, or water-immiscible liquids or solids as well as suspensions can be considered as active ingredients to be encapsulated. These are predominantly non-polar substances. Such hydrophobic substances are almost always lipophilic, i.e., they dissolve well in fat and oil. The terms “microcapsule” and “capsule” or “hydrophobic” and “lipophilic” are used synonymously within the context of the present invention.

In the context of the present description, the core material is preferably a hydrophobic active substance, i.e., a substance that has a specific effect or causes a specific reaction, for example, a drug, a pesticide, a cosmetic active ingredient, a food active ingredient, etc.

In a particularly preferred variant of the present invention, the at least one lipophilic or hydrophobic active ingredient is, in particular, a lipophilic or hydrophobic fragrance or aroma substance or a lipophilic or hydrophobic perfume oil or aroma (i.e. a mixture of fragrance substances or aroma substances, respectively), a cooling agent, a TRPV1 or a TRPV3 modulator, a substance that produces a pungent taste or a warmth or heat sensation on the skin or mucous membranes or a tingling sensation in the mouth or throat, or active ingredients with astringent effect, a pesticide, a biocide, an insecticide, a substance from the group of repellents, a food additive, a cosmetic active ingredient, a pharmaceutical active ingredient, a dye, a dye precursor; an agrochemical, a dye, a luminous paint, an optical brightener, a solvent, a wax, a silicone oil, a lubricant, a print coating for paper, or a mixture of two or more of the aforementioned active ingredients.

In a preferred embodiment according to the first and/or second aspect of the present invention, the microcapsules have a core material in the form of a hydrophobic single fragrant substance or single odorant substance, wherein the core material comprises at least one single fragrant substance or single odorant substance or mixtures thereof, selected from one or more of the following groups:

Extracts of natural raw materials and also fractions thereof or components isolated therefrom; single fragrance substances from a group of hydrocarbons; aliphatic alcohols; aliphatic aldehydes and acetals; aliphatic ketones and oximes; aliphatic sulfur-containing compounds; aliphatic nitriles; esters of aliphatic carboxylic acids; formates, acetates, propionates, isobutyrates, butyrates, isovalerates, pentanoates, hexanoates, crotonates, tiglinates and 3-methyl-2-butenoates of acyclic terpene alcohols; acyclic terpene aldehydes and ketones as well as their dimethyl and diethyl acetals; formates, acetates, propionates, isobutyrates, butyrates, isovalerates, pentanoates, hexanoates, crotonates, tiglinates and 3-methyl-2-butenoates of cyclic terpene alcohols; cyclic terpene aldehydes and ketones; cyclic alcohols; cyclic and cycloaliphatic ethers; cyclic and macrocyclic ketones; cycloaliphatic aldehydes; cycloaliphatic ketones; esters of cyclic alcohols; esters of cycloaliphatic carboxylic acids; aromatic hydrocarbons; araliphatic alcohols; esters of araliphatic alcohols and aliphatic carboxylic acids; araliphatic ethers; aromatic and araliphatic aldehydes; aromatic and araliphatic ketones; aromatic and araliphatic carboxylic acids and their esters; nitrogen-containing aromatic compounds; phenyl ethers and phenyl esters; heterocyclic compounds; lactones; and mixtures of the above active ingredients.

Specific examples of the abovementioned substances are known to the person skilled in the art. Fragrant substances or odorant substances and mixtures of two or more of said substances are chemical substances or compositions which are used to confer, impart and/or modulate a specific odour or odour impression. Preferably, said odour (impression) is considered as being pleasant.

In an alternative embodiment according to the present invention, the microcapsules according to the invention use a fragrance substance or a perfume oil, respectively, or an aroma substance or an aroma as the active ingredient to be encapsulated or as the core material. These are compositions containing at least one fragrance substance or one aroma substance. Such compositions, in particular mixtures of fragrance substances or perfume oils, preferably comprise two, three, four, five, six, seven, eight, nine, ten or more fragrance substances. The fragrance mixtures or perfume oils, respectively, are preferably selected from the group of extracts from natural raw materials, such as essential oils, concretes, absolutes, resins, resinoids, balsams, tinctures such as e.g., Ambergris oil; Amyris oil; Angelica seed oil; Angelica root oil; Anise oil; Valerian oil; Basil oil; Tree moss absolute; Bay oil; Mugwort oil; Benzoin resin; Bergamot oil; Beeswax absolute; Birch tar oil; Bitter almond oil; Savory oil; Bucco leaf oil; Cabreuva oil; Cade oil; Calmus oil; Camphor oil; Cananga oil; Cardamom oil; Cascarilla oil; Cassia oil; Cassie absolute; Castoreum absolute; Cedar leaf oil; Cedarwood oil; Cistus oil; Citronella oil; Citron oil; Copaiva balsam; Copaiva balsam oil; Coriander oil; Costus root oil; Cumin oil; Cypress oil; Davana oil; Dill herb oil; Dill seed oil; Eau de brouts absolute; Oak moss absolute; Elemi oil; Tarragon oil; Eucalyptus citriodora oil; Eucalyptus oil; Fennel oil; Spruce needle oil; Galbanum oil; Galbanum resin; Geranium oil; Grapefruit oil; Guaiac wood oil; Gurjun balsam; Gurjun balsam oil, Helichrysum absolute; Helichrysum oil; Ginger oil; Iris root absolute; Iris root oil; Jasmine absolute; Calamus oil; Chamomile oil blue; Chamomile oil Roman; Carrot seed oil; Cascarilla oil; Pine needle oil; Curly mint oil; Caraway seed oil; Labdanum oil; Labdanum absolute; Labdanum resin; Lavandin absolute; Lavandin oil; Lavender absolute; Lavender oil; Lemongrass oil; Lovage oil; Lime oil distilled; Lime oil pressed; Linal oil; Litsea cubeba oil; Bay leaf oil; Macis oil; Marjoram oil; Mandarin oil; Masso bark oil; Mimosa absolute; Musk grain oil; Musk tincture; Muscat sage oil; Muscat oil; Myrrh absolute; Myrrh oil; Myrtle oil; Clove leaf oil; Clove flower oil; Neroli oil; Olibanum absolute; Olibanum oil; Opopanax oil; Orange flower absolute; Orange oil; Origanum oil; Palmarosa oil; Patchouli oil; Perilla oil; Perubalsam oil; Parsley leaf oil; Parsley seed oil; Petitgrain oil; Peppermint oil; Pepper oil; Pimento oil; Pine oil; Poley oil; Rose absolute; Rosewood oil; Rose oil; Rosemary oil; Sage oil Dalmatian; Sage oil Spanish; Sandalwood oil; Celery seed oil; Spicy lavender oil; Star anise oil; Styrax oil; Tagetes oil; Fir needle oil; Tea tree oil; Turpentine oil; Thyme oil; Tolu balsam; Tonka absolute; Tuberose absolute; Vanilla extract; Violet leaf absolute; Verbena oil; Vetiver oil; Juniper berry oil; Wine yeast oil; Wormwood oil; Wintergreen oil; Ylang oil; Hyssop oil; Civet absolute; Cinnamon leaf oil; Cinnamon bark oil; and fractions thereof or ingredients isolated therefrom.

In a further variant of the invention according to the first and/or second aspect, aroma substances can also be encapsulated as a core material in the form of a single aroma, wherein the core material comprises at least one single aroma substance or mixtures thereof as active ingredient.

However, preferably a single fragrant substance or odorous substance (i.e., chemical compounds having a smell or odour and thus all natural and synthetic substances that impart an olfactorily perceptible odour) or alternatively a mixture of fragrant substances or odorous substances (so-called perfume oil) is used as the active ingredient enclosed by the microcapsule shell. More preferably, said substances and mixtures of substances are imparting a pleasant odour to a consumer product.

In a preferred variant of the present invention, all microcapsules of component a) are loaded with the same active ingredient(s), for example all of the microcapsules are comprising a fragrance substance and even more preferred the same fragrance substance.

Preferably, the composition according to the present invention does not contain free fragrant substances or perfume oil, i.e. the composition according to the present invention does not contain unencapsulated fragrance substances or perfume oil in order to achieve an effect as described herein when incorporated into solid consumer product formulations.

According to an alternative embodiment, the microcapsules according to component a) are encapsulating different classes of active ingredients or different substances among the same class of active ingredients. For example, some of the microcapsule comprise a specific fragrance substance while other microcapsules enclose another fragrance substance.

Thereby, preferably the core material is selected irrespective of the shell material.

Between 0.5% by weight to 10% by weight based on the total weight of the pastille of microcapsules can be incorporated into the composition, i.e. a single pastille of the composition according to the first and/or second aspect of the present invention comprises between 0.5% by weight to 10% by weight of microcapsule(s) as specified above. If the amount of component a) is kept within these ranges, a homogeneous distribution of the microcapsules within the pastille(s) can be achieved. Moreover, if the amount of component a) is within the above ranges the active ingredients can efficiently be incorporated into the final solid consumer product formulation due to an efficient protection by the surrounding polymeric matrix, i.e. the microcapsules do not break open during the preparation process of the solid consumer product and thus can effectively retain the encapsulated active ingredient(s) without loss, resulting in a significantly improved microcapsule performance as shown in the experimental section.

According to a preferred embodiment, the amount of the microcapsule(s) ranges from 0.5% by weight to 10% by weight based on the total weight of the pastille and even more preferred from 3% by weight to 8% by weight based on the total weight of the pastille.

The inventive compositions comprising the microcapsules encapsulating the active ingredient(s) are efficiently incorporated into a variety of solid consumer product formulations allowing for an efficient and targeted release of the active ingredient(s). Thereby the surrounding polymeric matrix acts as kind of a shock absorbing layer protecting the microcapsules from damage due to mechanical, chemical and/or thermal influences and thus premature release and degradation of the active ingredient(s) during the preparation process of the consumer product.

### Polymer or Polymer Matrix

As specified above, the compositions/pastilles according to the first and/or second aspect of the present invention comprise or consist of from 92% to 98% by weight or from 60% to 80% by weight based on the total weight of a pastille, respectively, of at least one polymer.

Suitable polymers according to the present invention are meltable polymers, such as for example polyethylene glycol (“PEG”) having a molecular weight from about 3,000 to 20,000 Da, preferably from about 4000 to 12000 Da, and even more preferred from about 5000 to 8000 Da.

Component b) can comprise either one specific polymer having a molecular weight within the ranges specified above, or two or more different polymers. Said different polymers can be either the same substance differing in their molecular weight within the specified ranges (such as PEG 3000 and PEG 4000), or can be different chemical substances having either the same or different molecular weights.

If the molecular weight of the polymer used as component b) is within the above specified range an efficient protection of the fragile microcapsules can be achieved allowing for an efficient incorporation in solid consumer product formulations.

In the context of the present invention, a “meltable” polymer is a plastic polymer material that becomes pliable or moldable at a certain elevated temperature and solidifies upon cooling.

The polymer used within the context of the present invention can be either a water-soluble or a water-insoluble polymer. According to an alternative embodiment component b) comprises a mixture of one or more water-soluble polymers, a mixture of one or more water-insoluble polymers, or a mixture of one or more water-soluble polymers and one or more water-insoluble polymers.

In a preferred variant of the present invention the melting point of the polymer according to the first and/or second aspect of the present invention (preferably having a molecular weight from about 3,000 to 20,000 Da) is preferably in the range from 25° C. to 100° C., and preferably from 40° C. to 80° C.

Preferably however, the polymer used in the context of the first and second aspect of the present invention as component b) is water-soluble. For example, when incorporated in textile care formulations such as powder detergents the polymeric protecting matrix will be dissolved releasing the microcapsules which thus can be applied to the textile.

Examples of suitable water-soluble polymers are for example polyvinyl alcohol, derivatives of polyvinyl alcohol (such as quaternary amine derivatives), polyethylene glycol (of varying molecular weight), polyvinyl pyrrolidone, (poly)acrylates, polyacrylic acid, acrylamides, polyamino acids and amine-functional polymers, saccharides and polysaccharides such as starch, modified starch, maltodextrins, carbohydrates, chitosan, and gum arabic, maleic anhydride copolymers, vinyl acids and ethers, styrenes, polystyrene sulfonates, ethylene glycol-propylene glycol block copolymers and mixtures thereof. Examples of suitable water-insoluble polymers include amongst others classical capsules with coating, waxes such as beeswax, sunflower seed wax, rice bran wax, carnauba wax, pinova wax, rapeseed wax, soywax, candelilla wax, jojoba oil, cork wax, guaruma wax, cotton wax, foxwax, peat wax, rose wax, jasmine wax, peetha-wax from wax gourd, myrtle wax (Myrica cerifera), wax fig wax, berry wax and mixtures of different waxes.

In a preferred embodiment according to the present invention, the polymer is preferably polyethylene glycol (also known as polyethylene oxide (PEO) or polyoxyethylene (POE)) having a molecular weight from about 3,000 to 20,000 Da, preferably from about 3,000 to 12,000 Da, and even more preferred from about 4,000 to 9,000 Da. According to an alternative preferred embodiment component b) comprises a mixture of two or more polyethylene glycols differing in their molecular weight.

Polyethylene glycol is a cheap, synthetic, hydrophilic, biocompatible polymer with widespread use in biomedical and other applications such as in pharmaceutical compositions as excipient or as laxative, in the chemical industry e.g. as lubricating coating, and in cosmetic formulations as a basis for creams or as dispersant. Polyethylene glycol is a flexible, water-soluble polymer having a low toxicity which can be incorporated into a broad variety of formulations and having the general formula: C₂ₙH₄ₙ₊₂Oₙ₊₁.

The compositions of the present invention according to the first aspect may comprise from 91% to 98% by weight based on the total weight of a pastille of the polymer (component b)), preferably from 92% to 98% by weight, and even more preferred from 92% to 96% by weight.

The compositions of the present invention according to the second aspect may comprise from 60% to 80% by weight based on the total weight of a pastille of the polymer (component b)), preferably from 92% to 98% by weight, and even more preferred from 92% to 96% by weight.

If the polymer specified above is comprised within said ranges in the compositions according to the present invention an efficient protection of the fragile microcapsules can be achieved.

According to another preferred variant of the first and second aspect of the present invention the polymer(s) exhibit(s) a specific solidification point: The minimum solidification point of the water-soluble polymer-like particles, i.e. the final pastilles, is preferably above 0° C., preferably above 20° C., and particularly preferably above 35° C., while the minimum solidification point of the non-water-soluble polymer-like particles is at above 0° C., preferably above 20° C., and particularly preferably above 35° C. Solidification points within said ranges allow a facilitated formation of the pastilles according to the present invention, without requiring low temperatures for the pastille preparation process and their subsequent processing.

### Adhesion Agent

Interestingly it was found that a combination of a polymer as specified above with an additional adhesion agent likewise results in an efficient protection of the microcapsules upon incorporation into solid consumer product formulations.

Thus, in a second aspect the present invention relates to a composition comprising or consisting of a microcapsule component (component a)), a polymer component (component b)) and an adhesion agent component (component c)) in specific ratios. Thereby the components b) and c) are forming the protective matrix surrounding, i.e. embedding the fragile microcapsules.

An adhesion agent according to the present invention is defined as a substance allowing for an increased adhesion of the microcapsules on external materials and their surfaces such as textiles (adhesion promoter). The adhesion is based on mechanical bonding, hydrogen bonding, chemical or covalent bonding, thermodynamic bonding, etc.

Preferably the adhesion agent used as component c) according to the second aspect of the present invention is a substance selected from the group consisting of fatty acids, fatty alcohols, fatty acid esters or mixtures thereof, such as natural and synthetic waxes. Thereby the composition according to the present invention can comprise one or a mixture of two or more of such adhesion agents as component c) according to the second aspect.

Further suitable adhesion agents according to the second aspect of the present invention are for example:

Long-chain, aliphatic, linear or branched, saturated or unsaturated carboxylic acids with 12 to 30 carbon atoms (fatty acids), in particular lauric acid (12:0), tridecanoic acid (13:0), myristic acid (14:0), pentadecanoic acid (15:0), palmitic acid (16:0), margaric acid (17:0), stearic acid (18:0), nonadecanoic acid (19:0), arachidic acid (20:0), heneicosanoic acid (21:0), behenic acid (22:0), lignoceric acid (24:0), cerotinic acid (26:0), montanic acid (28:0), and melissic acid (30:0); myristoleic acid (14:1), palmitoleic acid (16:1), margaroleic acid (17:1), petroselinic acid (18:1), oleic acid (18:1), elaidic acid (18:1), vaccenic acid (18:1), gadoleic acid (20:1), gondoic acid (20:1), cetoleic acid (22:1), erucic acid 822:1), nervonic acid (24:1), linoleic acid (18:2), alpha-linolenic acid (18:3), gamma-linolenic acid (18:3), calendulic acid (18:3), punicic acid (18:3), alpha-eloeostearic acid (18:3), beta-eloeostearic acid (18:3), stearidonic acid (18:4), arachidonic acid (20:4), eicosapentaenoic acid (20:5), docosatetraenoic acid (ADA) (22:4), docosapentaenoic acid (DPA-3) (22:5), docosahexaenoic acid (22:6), and tetracosahexaenoic acid (24:6), phytanic acid;
Long-chain, aliphatic, linear or branched, saturated or unsaturated primary alcohols with 12 to 30 carbon atoms (fatty alcohols), in particular lauryl alcohol (12:0), myristyl alcohol (14:0), palmityl alcohol (16:0), margaryl alcohol (17:0), stearyl alcohol (18:0), arachidyl alcohol (20:0), behenyl alcohol (22:0), lignoceryl alcohol (24:0), ceryl alcohol (26:0), montanyl alcohol (28:0), and melissyl alcohol (30:0); palmitoleyl alcohol (16:1), oleyl alcohol (18:1), elaedyl alcohol (18:1), linoleyl alcohol (18:2), gamma-linolenyl alcohol (18:3);
Esters of long-chain, aliphatic saturated carboxylic acids containing 12 to 30 carbon atoms with long-chain, aliphatic primary alcohols containing 12 to 30 carbon atoms, in particular lauryl palmitate, myricyl palmitate, cetylarachinate and stearyl behenate;
Animal and vegetable waxes, in particular wool wax, China wax, beeswax, sunflower seed wax, rice bran wax, carnauba wax, pinova wax, rapeseed wax, soy wax, candelilla wax, jojoba oil, cork wax, guaruma wax, cotton wax, flax wax, peat wax, rose wax, jasmine wax, peetha wax from wash gourd, and also myrtle wax (Myrica cerifera), wax fig wax, berry wax,
as well as mixtures of two or more of the aforementioned adhesion agents.

Of the carboxylic acids listed above, the saturated fatty acids are preferred. Most preferred in the context of the invention is the use of the animal and vegetable waxes specified above due to their lower melting points, which facilitate their incorporation into the composition.

According to another preferred embodiment, the at least one adhesion agent is selected from the group consisting of animal and/or vegetable waxes, and is preferably (natural) beeswax.

Surprisingly, it was found that such natural or synthetic animal and/or vegetable waxes can be efficiently incorporated into the composition according to the second aspect. Moreover, these substances allow for an improved adhesion of the microcapsules onto the surface of other materials such as textile fibres and simultaneously supports the protecting effect of the polymeric matrix.

The adhesion component is incorporated into the polymeric matrix.

Preferably the adhesion agent used as component c) according to the second aspect of the present invention is comprised in an amount of from 2% to 32% by weight based on the total weight of the pastille, preferably from 3% to 20% by weight, and more preferred from 4% to 17% by weight.

The addition of the adhesion agent(s) within said ranges allows for an improved balance between microcapsule protection and adhesion promotion.

### Plasticizer

The composition according to the first and/or second aspect can further comprise a certain amount of at least one plasticizer as additive. Thereby the compositions according to the present invention can comprise one or a mixture of two or more of such plasticizers.

A plasticizer is a substance that is used to increase the flexibility and plasticity of a specific substance or composition and can additionally decrease the viscosity as well as the friction of a substance or composition.

Suitable plasticizers are known to the person skilled in the art. Suitable plasticizers are for example water, polyethylene glycol, ortho phthalates, phthalate esters, trimellitates and esters thereof, (organo) phosphates, aliphatic dibasic acid esters, benzoate esters, polyesters, citrates, bioderived plasticizers based on epoxidized soybean oil, epoxidized linseed oil, castor oil, palm oil, other vegetable oils, starches, sugars and the like, chlorinated paraffins, alkyl sulfonic acid esters, dicarbonates, fatty acid esters.

Preferably the plasticizer(s) is/are selected from amongst others water or polyethylene glycol. The plasticizer water can origin for example from the manufacture process of the pastilles or can be added subsequently.

Preferably the plasticizer(s) used as additive(s) according to the first and/or second aspect of the present invention is/are comprised in an amount of from 0.5% to 10% by weight based on the total weight of the pastille, preferably from 2% to 8% by weight, and more preferred from 3% to 6% by weight.

The addition of the plasticizer(s) within said ranges allows for efficiently adjusting the viscosity and flexibility of the polymeric matrix resulting in a less brittle and more flexible matrix with improved buffering effect and thus better protection of the fragile microcapsules and a facilitated incorporation into solid consumer product formulations without altering the fundamental chemical character of the plasticized material.

### Structuring Agent

Additionally, it is possible to add one or more structuring agents to the compositions according to the first and/or second aspect of the present invention and preferably to the compositions according to the second aspect as specified above as additional component or additive. Thereby the composition according to the present invention can comprise one or a mixture of two or more of such structuring agents.

Said structuring agent(s) is/are preferably comprised in an amount ranging from 0.1% to 10% by weight based on the total weight of a pastille, preferably from 1% to 8% by weight and even more preferred from 2% to 7% by weight.

Suitable structuring agents are commercially available emulsifiers known in the art and esters such as isopropyl myristate.

The addition of such structuring substance results in an improved sphere formation during droplet formation. Based thereon the incorporation of the microcapsules/pastilles into the final product formulation is enhanced in a homogeneous manner. These substances are for example an additive that increases the viscosity of the composition to a thick liquid, semi-solid, or solid state.

Additionally, the compositions according to the present invention can comprise further suitable additives if required.

### Composition/Pastille

The compositions according to the first and second aspects of the present invention comprise or consist of at least one pastille having the composition specified above. Preferably the composition is present as a plurality of pastilles. Alternatively, the composition is present as a single pastille of the above-specified composition.

In a preferred embodiment according to the present invention, the compositions and consequently the pastilles according to the first and second aspect of the present invention have a solidification point from 25° C. to 100° C., preferably from 30° C. to 90° C., more preferably from 35° C. to 85° C. and most preferred from 40° C. to 75° C.

A solidification point within this range allows for a facilitated handling of the compositions according to the present invention and the pastilles thereof.

According to another preferred embodiment the composition according to the first and/or second aspect of the present invention and consequently the corresponding pastille has a viscosity in the range from 500 mPas to 1,000 mPas at a temperature of 70° C. and a shear pressure of 6.4 Pa, preferably a viscosity in the range from 760 mPas to 980 mPas, and even more preferred a viscosity in the range from 800 mPas to 950 mPas.

For compositions according to the present invention the viscosity is even more preferred in the range from 750 mPas to 1,000 mPas at a temperature of 70° C. and a shear pressure of 6.4 Pa, preferably a viscosity in the range from 760 mPas to 980 mPas, and even more preferred a viscosity in the range from 800 mPas to 950 mPas. If the viscosity of the compositions is within the above ranges, the composition is considered “solid” at room temperature.

Thus, preferably, the composition according to the present invention is preferably a solid composition.

If the viscosity of the compositions is within the above ranges processing of the compositions and their incorporation into solid consumer product formulations is facilitated. Additionally, if the viscosity is within the above ranges an ideal balance between microcapsule protection and microcapsule performance can be achieved.

Based on another preferred variant of the present invention, the compositions according to the first and/or second aspect of the present invention and consequently the corresponding pastilles, have a Young's modulus E of higher than 100 Pa and less than 1 GPa, preferably from 1 kPa to 100 MPa, and even more preferred from 10 kPa to 1 MPa.

The Young's modulus E describes the tensile or compressive stiffness of a solid but elastic material when the force is applied lengthwise based on the tensile/compressive stress (force per unit area) and axial strain (proportional deformation) in the linear elastic region of a material.

An elasticity within said ranges allows for ideal balance between microcapsule protection and their efficient incorporation into solid consumer product formulations while allowing for excellent release properties.

In another preferred variant the water content in the composition according to the first and second aspect of the present invention and thus of the corresponding pastilles is from 2% to 12% by weight based on the total weight of a pastille. Preferably the water content is in the range from 3% to 9% by weight and even more preferred in the range from 4% to 8% by weight.

Such amounts of water are advantageous in terms of flexibility as the water component can function as plasticizer and thus is valuable in view of an improved buffering effect and thus better protection of the fragile microcapsules and a facilitated incorporation into solid consumer product formulations without altering the fundamental chemical character of the plasticized material. Higher amounts of water will decrease the viscosity too much making an incorporation into solid formulations more difficult while to low water contents make the pastille material to brittle for an incorporation.

The abovementioned properties of the compositions according to the first and second aspect of the present invention can be adjusted based on the specific amounts of the components, the molecular weight of the polymer as well as the optional addition of additives such as the plasticizers and structuring agents described above.

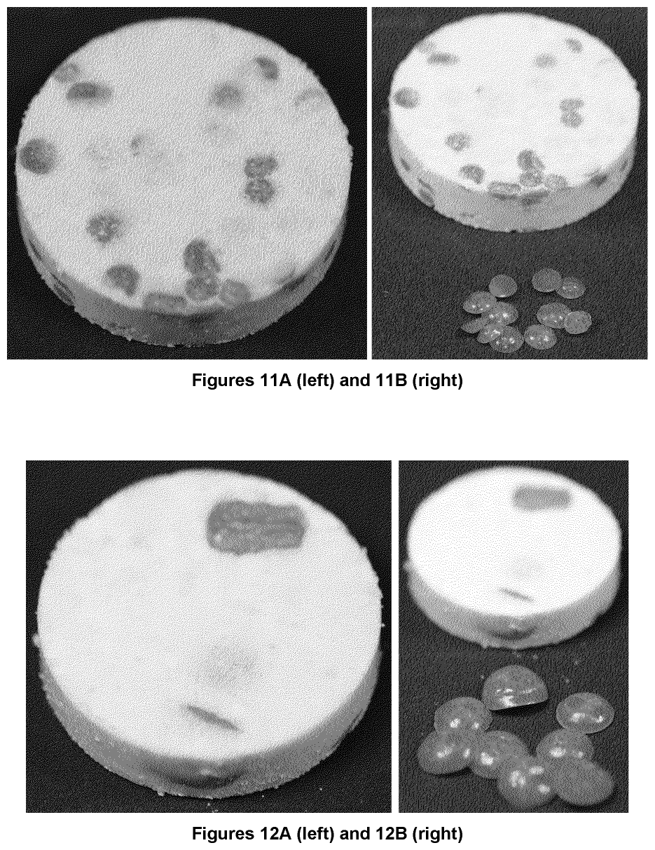

It is additionally advantageous if the particle size of the pastille(s) of the compositions according to the first and/or second aspect of the present invention is from 1 μm to 1 cm, preferably from 100 μm to 7 mm and even more preferred from 500 μm to 5 mm in order to achieve an ideal distribution oft the pastilles within the final solid consumer product formulation and thus in turn of the microcapsules embedded therein. Too big pastilles result in an inhomogeneous distribution (see FIGS. 11 and 12).

In a further preferred variant of the present invention the ratio between the size of the microcapsules and the size of the final granules/pastilles is in the range of from 1:5 to 1:200, preferably from 1:10 to 1:100.

Said pastilles or granules are preferably present in the shape of tablets, pills, spheres, and the like, having a spherical, hemispherical, compressed hemispherical, lentil shaped, and oblong shape and can be prepared by any method known in the art by those skilled in the art.

Moreover, the composition of the invention and the pastilles thereof may further comprise one or more ingredients which improve the visual aesthetics of the final product such as colourants. In addition, the composition can comprise further additive and/or active ingredients such as optical brighteners, cleaning agents, laundry actives and the like.

### Process for the Preparation of the Inventive Compositions

According to a third aspect the present invention relates to a process for the preparation of a composition comprising or consisting of a plurality of pastilles comprising the following steps of:
(i) providing at least one microcapsule or an aqueous dispersion comprising at least one microcapsule;
(ii) melting of at least one polymer and optionally an adhesion agent;
(iii) mixing the at least one microcapsule into the polymer melt of step (ii);
(iv) forming pastilles by dripping the mixture of step (iii); and
(v) optionally cooling;
   optionally further comprising the step of adding at least one plasticizer and/or a structuring agent in or after step (ii) or (iii).

In the context of the present invention, the components specified above in the context of the first and second aspect likewise apply to the subject of the third aspect, i.e. the terms “microcapsule”, “polymer”, “adhesion agent”, “plasticizer”, “pastille”, “structuring agent” etc. as well as the corresponding amounts and properties as specified above.

The process according to the third aspect as specified herein is suitable for the preparation of the compositions according to the first and second aspect of the present invention based on the corresponding adjustment of the required amounts.

In a first step (i) of the process according to the present invention microcapsules as specified herein are provided. Said microcapsules can either be present as such or alternatively in the form of an aqueous dispersion comprising one or more microcapsules and preferably a plurality of microcapsules.

Preferably, the microcapsules are dispersed in an aqueous phase, microcapsules according to the invention dispersed in an aqueous phase, preferably wherein the microcapsule concentration within the aqueous phase is greater than 5% and less than 70% by weight, preferably greater than 20% and less than 60% by weight, most preferably greater than 30% and less than 50% by weight based on the weight of the dispersion.

The provision of microcapsules in the form of a dispersion is advantageous in terms of a more homogeneous distribution of the microcapsules within the final pastilles. Additionally, the comprised water of the continuous or outer phase of the dispersion can serve as plasticizer helping to improve the final product properties as specified above. allowing for an efficient adjustment of the viscosity and flexibility of the polymeric matrix resulting in a less brittle and more flexible matrix with improved buffering effect and thus better protection of the fragile microcapsules and a facilitated incorporation into solid consumer product formulations without altering the fundamental chemical character of the plasticized material.

Therefore, preferably the microcapsule(s) is/are provided in the form of an aqueous dispersion in step (i) of the process according to the third aspect of the present invention.

In a subsequent step (ii) of the process according to the invention, the at least one polymer is melted. Depending on the approach an adhesion agent can be added which is heated and melted together with the polymer as specified herein. Alternatively, both substances can be melted separately and subsequently the resulting corresponding melts combined and homogenized.

In order to achieve a homogeneous distribution of microcapsules within the polymeric matrix it is further preferred to use a polymer as specified herein, and e preferably polyethylene glycol, wherein the melting point of the at least one polymer, and preferably of the polyethylene glycol, is in the range from 25° C. to 100° C., and preferably from 40° C. to 80° C.

Thus, according to a further preferred variant of the present invention the melting point of the polymer according to the first, second and/or third aspect of the present invention preferably having a molecular weight from about 3,000 to 20,000 Da is preferably in the range from 25° C. to 100° C., and preferably from 40° C. to 80° C. in order to efficiently and homogeneously embed the microcapsules within the polymeric matrix.

In a preferred variant of the process according to the invention correspondingly the melting step (ii) is conducted at a temperature in the range from 25° C. to 100° C., preferably from 40° C. to 80° C., and even more preferred at 80° C. in order to achieve a homogeneous distribution of microcapsules in the polymeric buffering matrix.

The at least one microcapsule or microcapsule dispersion is mixed into the polymer melt of step (ii) and the resulting mixture homogenized in a further process step (iii) in order to obtain a homogeneous distribution of discrete microcapsules within the melt.

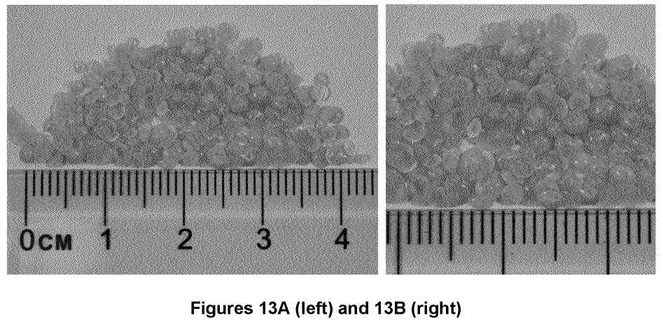

Finally, in a subsequent step (iv) pastilles are formed by dripping the mixture of step (iii) using state-of-the-art pastillation processes known to those skilled in the art. Pastillation is a particle forming process, where usually a pastille is formed using a droplet former, followed by cooling of the as-obtained discrete particles. Processes for the preparation of pastilles or granules are commercially used methods. Moreover, up-scaling by dropping using a pulse generator in air or vegetable oil also showed good results. FIG. 13 shows exemplarily prepared pastilles of the composition according to the present invention.

Optionally the obtained granules are solidified in an additional cooling step (v).

If required, at least one plasticizer and/or a structuring agent can be added in or after step (ii) and/or (iii).

In order to achieve a homogeneous distribution of microcapsules within the protecting matrix it is advantageous to use microcapsules having a particle size ranging from 150 μm to 5 mm, preferably in the rage from 500 μm to 3 mm.

Thus, according to a preferred variant of the present invention the particle size of the microcapsule(s) according to the first, second and/or third aspect of the present invention is preferably in the range from 150 μm to 5 mm, preferably in the rage from 500 μm to 3 mm.

Additionally, in a further aspect of the present invention relates to compositions prepared according to the inventive process.

### Consumer Products

Additionally, in another aspect, the present invention relates to the use of the compositions according to the invention for the preparation of consumer product formulations. The terms “consumer product formulation” and “consumer product” are used synonymously within the context of the present invention.

Thereby, any of the abovementioned compositions is suitable for the preparation of consumer product formulations, i.e. compositions according to the first aspect of the present invention, compositions according to the second aspect of the present invention and/or compositions obtained based on the process according to the third aspect of the present invention.

Particular advantages of the inventive pastille compositions were found in view of the preparation of solid consumer products such as powder detergents, laundry soaps, solid fabric softeners, deo sticks and the like. Thus, preferably the consumer product according to the present invention is a solid consumer product.

Therefore, in another preferred variant the present invention also relates to the use of the composition according to the present invention for the preparation of consumer products and formulations thereof, wherein said consumer product or consumer product formulation is a solid product such as a textile care product (such as a powder detergent, laundry soap, solid fabric softener), a solid soap, a household product (such as a solid WC-cleaner, solid all-purpose cleaners, powdered carpet cleaners, powdery washing agents for washing dishes or for cleaning various surfaces), a personal care product (such as a deodorant, soap), scent boosters, fragrance enhancers, a pharmaceutical product, and mixtures thereof.

Finally, in another aspect the present invention relates to the consumer product or consumer product formulation as such comprising or consisting of the composition according to the invention, i.e. the composition according to the first aspect of the present invention, the composition according to the second aspect of the present invention and/or the composition obtained based on the process according to the third aspect of the present invention.

The inventive compositions allow for the efficient incorporation of active ingredients into solid product formulations allowing for an efficient and targeted release of said active ingredients.

### EXAMPLES

Hereinafter, the present invention is described in more detail and specifically with reference to the examples, which however are not intended to limit the present invention.

### Example 1: Composition Comprising Microcapsules in Laundry Soap Without Adhesion Agent (Composition I)—Sensory Evaluation

According to a first example a composition comprising microcapsules was prepared as follows: In a first step core-shell microcapsules comprising a melamine-formaldehyde-based shell material and a fragrance core were provided (microcapsule size: 10 to 50 μm). The microcapsules were prepared according to the state of the art. Subsequently, the polymer polyethylene glycol (molecular weight: 5400 Da to 6600 Da) was melted at a temperature of 80° C. and the microcapsules mixed with the as-obtained polymer melt. In a next step, the viscous mixture was dripped a using state-of-the-art pastillation process by dripping the viscous material through small openings in order to form a plurality of pastilles having an average particle size of approximately 2 mm in which the microcapsules are embedded in a polyethylene glycol matrix, followed by cooling for solidification of the pastilles. The portion of the polyethylene glycol of the final pastilles was about 98% by weight, 95% by weight, and 92% by weight based on the total weight of each pastille, respectively.

The sensory evaluation was performed as follows: Once the composition according to the present invention was prepared the as-obtained microcapsule-composition was combined with commercially available laundry soap in the form of soap particles and the resulting mixture passed through an extruder and the final soap cut into pieces. The final dosage of the microcapsules in 30 g of the laundry soap was 0.4% by weight.

The as-prepared laundry soap was then applied to damp fabrics (terry cloth) by rubbing. After rinsing the fabrics were air dried at room temperature.

A corresponding laundry soap not comprising the inventive composition comprising perfumed microcapsules was used as reference. As a second reference, a corresponding laundry soap was used which comprised the pure melamine-formaldehyde-based shell material and a fragrance core but no surrounding polyethylene glycol matrix. In a third reference the capsules were prepared by not incorporating them into a matrix as with the pastilles specified above, but by coating them individually with polyethylene glycol (thickness of the coating approximately 5 to 25 nm).

All microcapsules have a particle size in the range of 10-50 μm.

The fragrance release was carried out in three steps. The first step describes the smelling of an untreated cloth. The second step describes the smelling of a lightly kneaded cloth; for this purpose, the cloth was subjected to slight mechanical stress by moving it back and forth between the hands several times, causing the capsules to break. The third step describes the smelling after the cloths were rubbed strongly and thus the capsules broke. After each step, the fragrance intensity was evaluated.

8 to 9 test persons assessed the fragrance intensity on a scale of 1 (no odour) to 9 (very strong odour).

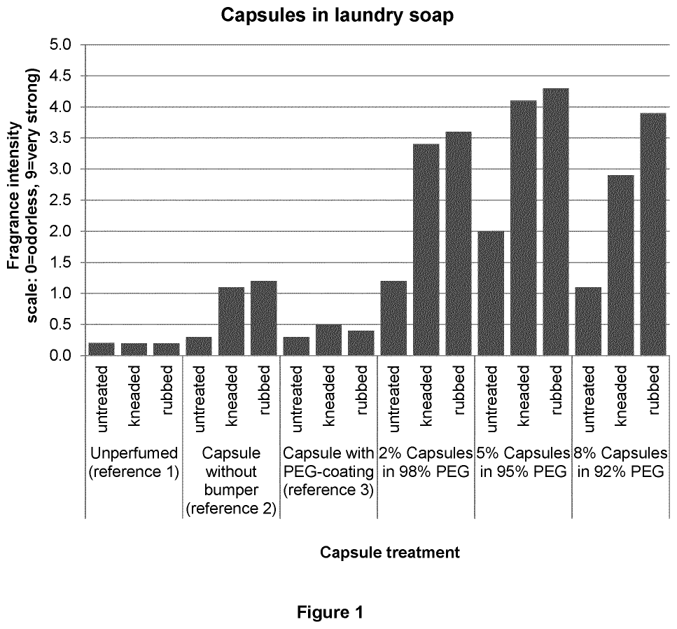

The results of the sensory evaluation are shown in FIG. 1.

The samples comprising the composition according to the present invention comprising the core-shell microcapsules embedded in a polyethylene glycol matrix show a considerably increased performance indicating an efficient protection of the fragile microcapsules within the solid product formulation while simultaneously allowing for an efficient release of the active ingredient. The polymer matrix serves as a shock absorbing layer in the production process of the consumer product. Samples not containing such a shock absorbing layer and such comprising a mere coating do not perform as most of the microcapsules are already destroyed during the preparation of the consumer product.

### Example 2: Composition Comprising Microcapsules in Laundry Soap With Adhesion Agent (Composition II)—Sensory Evaluation

According to a second example a composition comprising microcapsules was prepared as follows: In a first step core-shell microcapsules comprising a melamine-formaldehyde-based shell material and a fragrance core were provided (microcapsule size: 10 to 50 μm). The microcapsules were prepared according to the state of the art. Subsequently, the polymer polyethylene glycol (molecular weight: 5400 Da to 6600 Da) and beeswax as adhesion agent were melted at a temperature of 80° C. and the microcapsules mixed with the as-obtained polymer melt. The ratio between the polyethylene glycol and the beeswax was 75% by weight to 17% by weight based on the total pastille weight. In a next step, the viscous mixture was dripped a using state-of-the-art pastillation process by dripping the viscous material through small openings in order to form a plurality of pastilles having an average particle size of approximately 2 mm in which the microcapsules are embedded in a polyethylene glycol-beeswax matrix, followed by cooling for solidification of the pastilles. The polyethylene glycol-beeswax mixture of the final pastilles was about 92% by weight based on the total weight of each pastille.

Once the composition according to the present invention was prepared the as-obtained microcapsule-composition was combined with commercially available laundry soap in the form of soap particles and the resulting mixture passed through an extruder and the final soap cut into pieces. The final dosage of the microcapsules in 30 g of the laundry soap was 0.4% by weight.

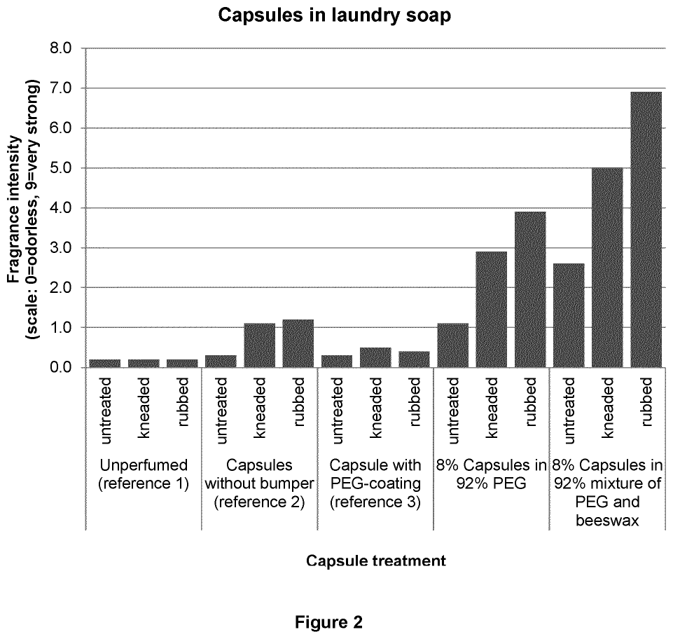

The sensory evaluation was performed according to example 1. The results of the sensory evaluation are shown in FIG. 2.

A comparison of samples comprising the a-prepared protected microcapsules compared to non-perfumed samples, samples comprising non-protected microcapsules as well as samples comprising merely coated microcapsules shows a significantly increased performance. Additionally, it was observed, that the combination of the polymer (polyethylene glycol) with an adhesion agent (beeswax) results in a considerable performance improvement compared to composition comprising a pure polyethylene glycol matrix, indicating an improved show absorbing and protective effect.

### Example 3: Composition Comprising Microcapsules in Tabletted Form Without Adhesion Agent (Composition I)—Sensory Evaluation

A plurality of pastilles in which melamine-formaldehyde-based perfume microcapsules are embedded in a polyethylene glycol matrix was prepared according to the process described in Example 1.

Subsequently, a powdered mixture of sodium sulfate and bentonite was provided and mixed with the as-prepared pastilles. After homogenization the mixture was pressed into tablets at 100 kN. The final dosage of the microcapsules in 10 g of the final tablet was 1% by weight.

The corresponding reference samples were prepared analogously: A corresponding tablet not comprising the inventive composition comprising perfumed microcapsules was used as reference. As a second reference, a corresponding tablet was used which comprised the pure melamine-formaldehyde-based shell material and a fragrance core but no surrounding polyethylene glycol matrix. In a third reference coated microcapsules were used.

In order to treat the fabric, liquid detergent and the as-prepared tablets were added into the laundry drum and the cloths washed at 30° C. followed by subsequent air-drying at room temperature. The as-prepared tablets were used as solid fabric softener.

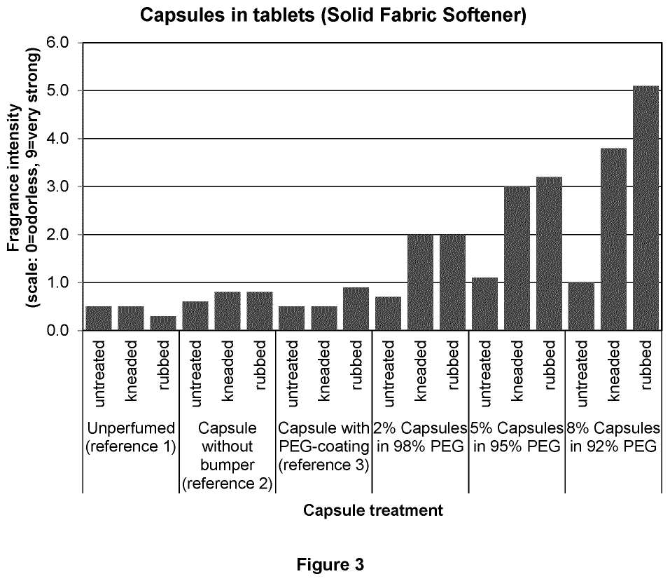

The sensory evaluation was performed according to example 1. The results of the sensory evaluation are shown in FIG. 3.

Example 3 likewise shows an improvement in the performance for solid product formulations comprising the inventive composition with a shock-absorbing layer around the fragile microcapsules allowing for the effective incorporation in said product without negatively influencing its release behaviour.

Moreover, surprisingly, higher loaded particles (92% by weight polyethylene glycol and 8% by weight of microcapsules per pastille) are sensory better compared to lower loaded particles (98% by weight polyethylene glycol and 2% by weight of microcapsules per pastille) at the same concentration in the final product. This shows that the incentive composition allows for an increase in the number of incorporated microcapsules.

### Example 4: Composition Comprising Microcapsules in Tabletted Form With adhesion agent (composition II)—Sensory evaluation

Example 4 was prepared and performed in accordance the preceding samples based on Example 3. However, in contrast to Example 3, the polymer matrix embedding the core-shell microcapsules additionally comprises a beeswax-component analogous to Example 2.

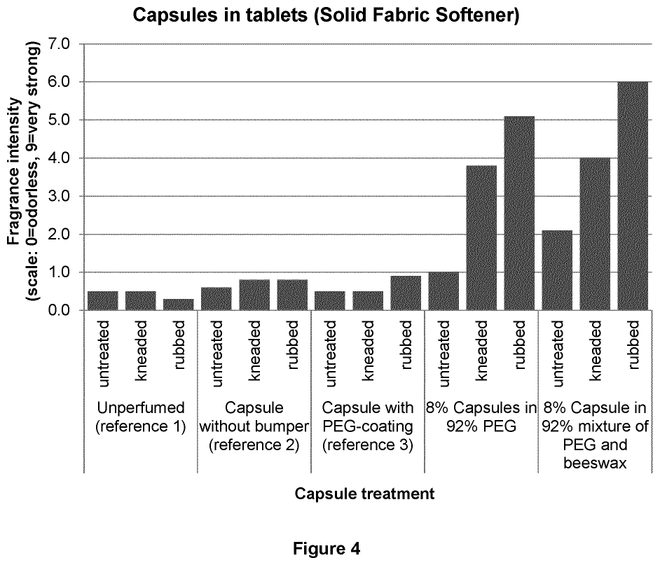

The results of the sensory evaluation are shown in FIG. 4.

The sensory evaluation revealed that both samples comprising capsules with polyethylene glycol only and the mixture with the adhesion agent beeswax show a significant improvement in the performance compared to the comparative samples.

### Example 5: Composition Comprising Microcapsules in Powder Detergent Without Adhesion Agent (Composition I)—Sensory Evaluation

A plurality of pastilles in which melamine-formaldehyde-based perfume microcapsules are embedded in a polyethylene glycol matrix was prepared according to the process described in Example 1.

Subsequently, a commercially available powder deterged was mixed with the as-prepared pastilles and homogenized. The final dosage of the microcapsules in 40 g of the laundry soap was 0.5% by weight.

Finally, the product was added to the laundry machine and the and the cloths washed at 30° C. followed by air-drying at room temperature.

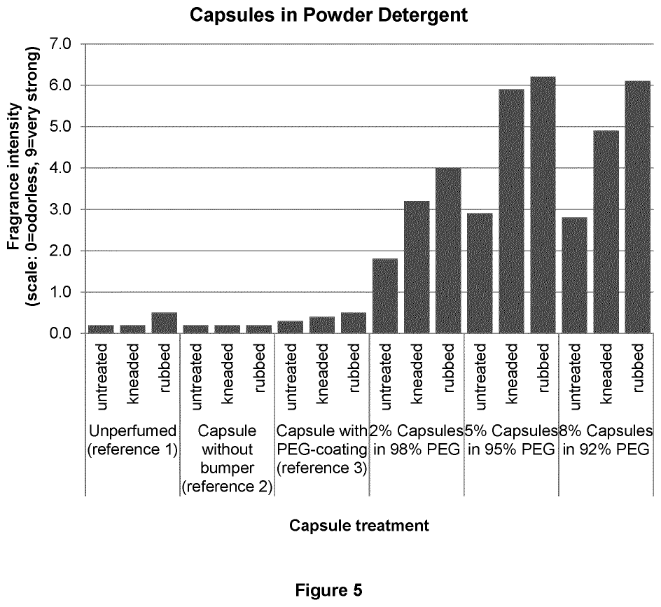

The sensory evaluation was performed according to example 1. The results of the sensory evaluation are shown in FIG. 5.

All samples show a significant improvement in the performance based on a buffer function of the polymer matrix in the consumer product preparation process. Non-protected microcapsules are destroyed upon mixing and homogenization with the powder detergent and are thus not suitable for the preparation of a perfumed powder detergent.

### Example 6: Composition Comprising Microcapsules in Powder Detergent With Adhesion Agent (Composition II)—Sensory Evaluation

According to another example, a composition according to the present invention comprising a plurality of pastilles was prepared, processed and analysed in accordance with Example 5, wherein the polymeric matrix additionally comprises an adhesion agent (beeswax).

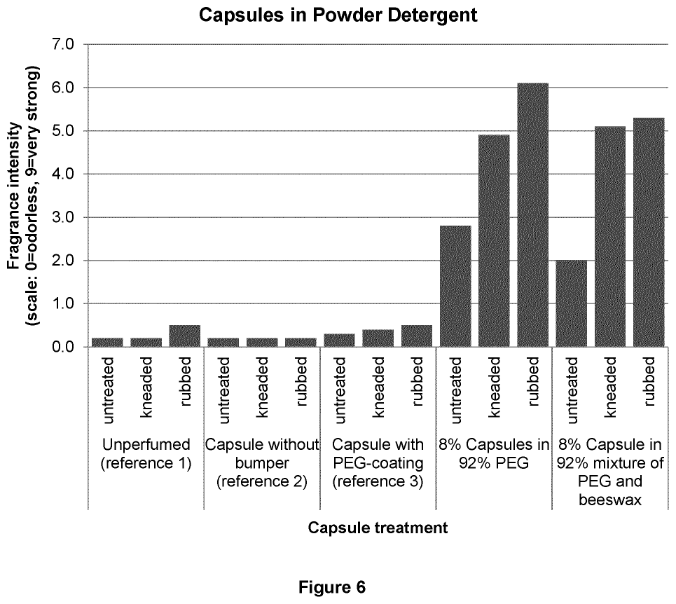

The results of the sensory evaluation are shown in FIG. 6.

FIG. 6 indicates, that depending on the formulation, the addition of the adhesion agent leads to a comparable sensory performance. In any case the performance of the perfumed powder detergent is increased by a factor of 5 to 6 compared to comparative powder detergents not containing a perfume composition according to the present invention.

### Example 7: Composition Comprising Biodegradable Microcapsules in Laundry Soap Without Adhesion Agent (Composition I)—Sensory Evaluation

Example 7 corresponds to Example 1 specified above. However, instead of core-shell microcapsules comprising a melamine-formaldehyde-based shell material and a fragrance core, biodegradable microcapsules comprising a biodegradable isocyanate—based shell material and a fragrance core were used. Thereby the shell material of the microcapsules used was based on the polymerization of 1,3-bis(6-isocyanatohexyl)-1,3-diazetidine-2,4-dione and bis (isocyantomethyl) cyclohexane with gelatin and maltodextrin.

Within the scope of the present invention microcapsules are considered biodegradable if the shell material of said microcapsule is degradable according to the OECD 310 F standard.

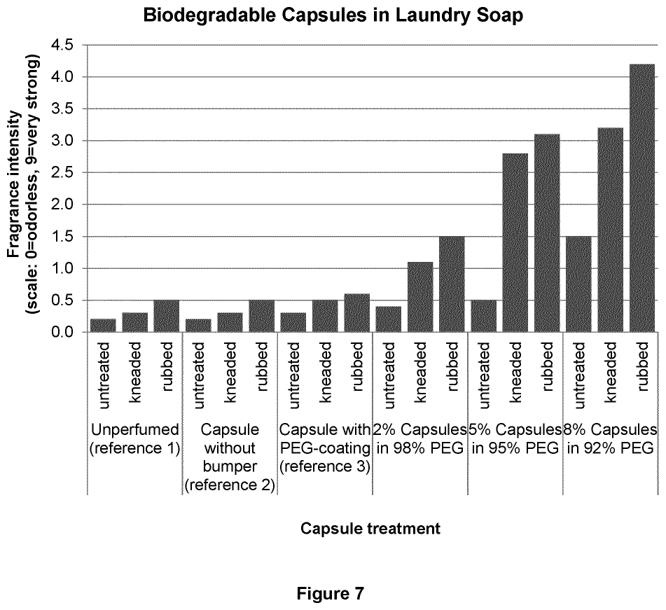

The results of the sensory evaluation are shown in FIG. 7.

The results indicate that the present composition is suitable independent of the microcapsule-composition as such and allows also for the efficient incorporation of biodegradable microcapsules into solid consumer product formulations such as laundry soap.

### Example 8: Composition Comprising Biodegradable Microcapsules in Laundry Soap With Adhesion Agent (Composition II)—Sensory Evaluation

According to another example, a composition according to the present invention comprising a plurality of pastilles was prepared, processed and analysed in accordance with Example 7, wherein the polymeric matrix additionally comprises an adhesion agent (beeswax).

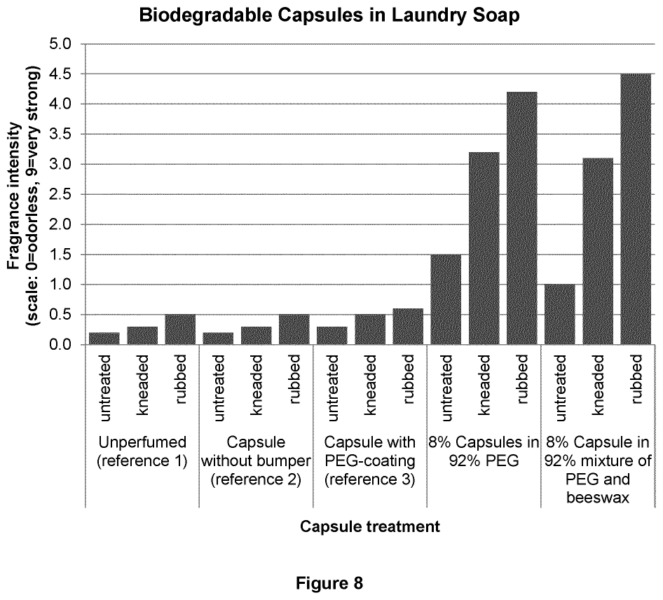

The results of the sensory evaluation are shown in FIG. 8.

FIG. 8 indicates that the performance of a perfumed solid laundry soap is increased by a factor of 4 to 5 compared to comparative laundry soaps not containing a perfume composition according to the present invention, when using either a pure polyethylene glycol matrix as well as a polyethylene glycol-beeswax matrix.

### Example 9: Composition Comprising Isocyanate-Based Microcapsules in Powder Detergent Without Adhesion Agent (Composition I)—Sensory Evaluation

Example 9 corresponds to Example 5 specified above. However, instead of core-shell microcapsules comprising a melamine-formaldehyde-based shell material and a fragrance core, microcapsules comprising a polyurea-based shell material being the reaction product of a polyisocyanate and a polyamine encapsulating a fragrance core were used. Thereby the shell material of the microcapsules used was based on the reaction of hexamethylene diisocyanate and methyl diphenyl diisocyanate with guanidinium carbonate.

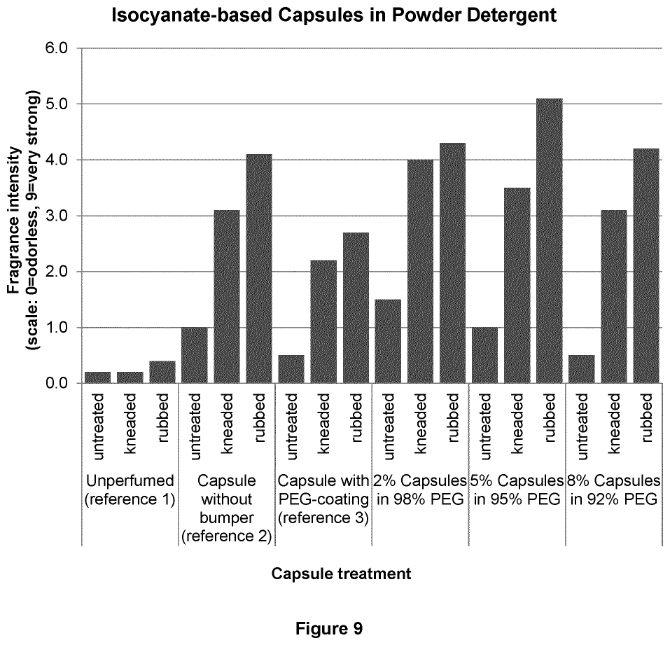

The results of the sensory evaluation are shown in FIG. 9.

As can be seen from FIG. 9, the embedding of microcapsules into a protecting polymer matrix allows for the more stable incorporation of microcapsules into solid consumer product formulations resulting in a visible gain in sensory performance.

### Example 10: Composition Comprising Isocyanate-Based Microcapsules in Powder Detergent With Adhesion Agent (Composition II)—Sensory Evaluation

According to another example, a composition according to the present invention comprising a plurality of pastilles was prepared, processed and analysed in accordance with Example 9, wherein the polymeric matrix additionally comprises an adhesion agent (beeswax).

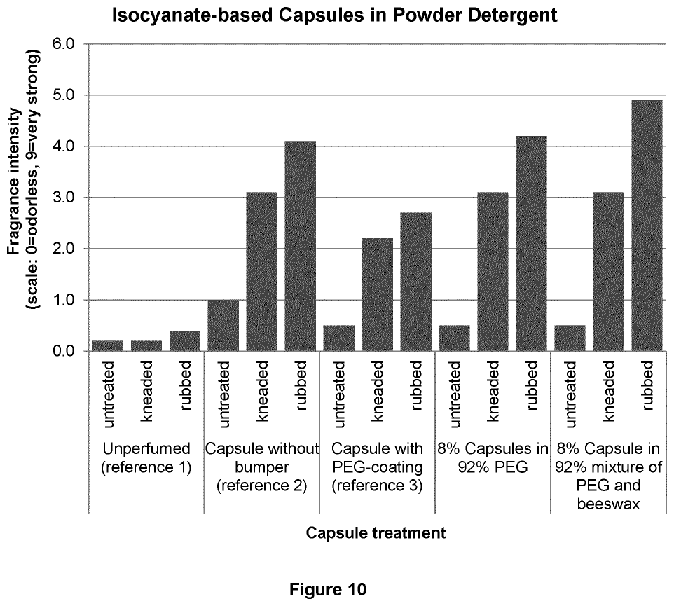

The results of the sensory evaluation are shown in FIG. 10.

The results show that the use of microcapsules embedded in a polymer-adhesion agent-matrix result in an even more improved performance of the consumer product.

1. A composition comprising at least one pastille, wherein said pastille comprises:
(a) from 0.5% to 10% by weight of at least one microcapsule encapsulating an active ingredient; and
(b) from 92% to 98% by weight of a polymer;
based on the total weight of the pastille.

2. The composition according to claim 1, wherein said at least one pastille further comprises:
(c) from 0.5% to 10% by weight of at least one plasticizer, based on the total weight of the pastille.

3. A composition comprising at least one pastille, wherein said pastille comprises:
(a) from 0.5% to 10% by weight of at least one microcapsule encapsulating an active ingredient;
(b) from 60% to 80% by weight of at least one polymer; and
(c) from 2% to 32% by weight of at least one adhesion agent;
based on the total weight of the pastille.

4. A composition according to claim 3, wherein said at least one pastille further comprises:
(d) from 0.5% to 10% by weight of at least one plasticizer; and/or
(f) from 0.1% to 10% by weight of a structuring agent,
based on the total weight of the pastille.

5. A composition according to claim 1, wherein the at least one polymer has a molecular weight from about 3,000 to 20,000 Da.

6. A composition according to claim 1, wherein the shell of the at least one microcapsule comprises a shell material selected from the group consisting of sol-gel polymer, polyacrylate, polyacrylamide, poly(acrylate-co-acrylamide), polyurea, polyurethane, polypeptide, polysaccharide, polyphenolic polymers, poly(melamine-formaldehyde), poly(resorcinol-formaldehyde), poly(urea-formaldehyde), and combinations thereof.

7. The composition according to claim 3, wherein the at least one adhesion agent is selected an animal and/or vegetable wax.

8. A process for the preparation of a composition comprising a plurality of pastilles, comprising the following steps of:
(i) providing at least one microcapsule or an aqueous dispersion comprising at least one microcapsule;
(ii) melting at least one polymer and optionally an adhesion agent;
(iii) mixing the at least one microcapsule into the polymer melt of step (ii);
(iv) forming pastilles by dripping the mixture of step (iii); and
(v) optionally cooling;
and optionally further comprising the step of adding at least one plasticizer and/or a structuring agent in or after step (ii) or (iii).

9. The process according to claim 8, wherein the melting point of the at least one polymer is in the range from 25° C. to 100° C.

10. The process according to claim 8, wherein the melting step (ii) is conducted at a temperature in the range from 25° C. to 100° C.

11. The process according to claim 8, wherein the particle size of the microcapsule(s) is in the range from 150 μm to 5 mm.

12. The process according to claim 8, wherein the microcapsule(s) is/are provided in the form of an aqueous dispersion.

13. A consumer product formulation comprising the composition according to claim 1.

14. The consumer product formulation of claim 13, wherein the formulation is a solid product, a solid soap, a household product, a personal care product, a pharmaceutical product, or a mixture thereof.

15. (canceled)

16. The composition according to claim 1 wherein the active ingredient is a fragrance or fragrance mixture and wherein the polymer is polyethylene glycol.

17. The composition according to claim 2 wherein the plasticizer is water or polyethylene glycol.

18. The composition according to claim 3 wherein the active ingredient is a fragrance or fragrance mixture, the polymer is polyethylene glycol, and the adhesion agent is selected from the group consisting of fatty acids, fatty alcohols, and fatty acid esters or mixtures thereof.

19. The composition according to claim 4 wherein the plasticizer is water or polyethylene glycol and wherein the structuring agent is selected from the group consisting of esters and other emulsifiers.

20. The process according to claim 10 wherein the melting step (ii) is conducted at a temperature from 40° C. to 80° C.

21. The consumer product formulation according to claim 14 wherein the solid product is a powder detergent, laundry soap, or solid fabric softener and the personal care product is a deodorant or soap.
